(19) United States
(12) Patent Application Publication
(10) Pub. No.: US 2002/0016307 A1
(43) Pub. Date: Feb. 7, 2002

(54) Pharmaceutical composition with both a lipase inhibitor and a lipophilic polysaccharide and an improved method for treating adiposity

(76) Inventor: John Jason Gentry Mullins, San Francisco, CA (US)

Correspondence Address: Robert G. Lev, 4766 Michigan Boulevard, Youngstown, OH, 44505, US

(21) Appl. No.: 09/901,166

(22) Filed: Jul. 9, 2001

Related U.S. Application Data
(63) Continuation-in-part of application No. 09/698,307, filed on Oct. 27, 2000.
Application No. 09/698,307 is a continuation-in-part of application No. 09/618,328, filed on Jul. 18, 2000, now abandoned.
Application No. 09/618,328 is a continuation-in-part of application No. 09/431,551, filed on Oct. 29, 1999, now Pat. No. 6,235,305.
Application No. 09/618,328 is a continuation-in-part of application No. 09/428,149, filed on Oct. 27, 1999.
Application No. 09/618,328 is a continuation-in-part of application No. PCT/US00/29641, filed on Oct. 27, 2000.

(51) Int. Cl. A61K 31/732; A61K 31/729; A61K 31/722; A61K 31/715

(52) U.S. Cl. 514/55; 514/54

(57) ABSTRACT

An improved antiadiposity anti-hypercholesterolemic dietary composition and method comprising the combination of a lipase inhibitor and a lipophilic polysaccharide, preferably a deacylated or sulfated polysaccharide. The lipase inhibitor and polysaccharide may be administered in the same dosage form or may be administered separately within close time proximity. The combination of these two active ingredients and method utilizing the combination give surprisingly improved synergistic results over the individual administration of the two active ingredients.

The present application is a continuation-in-part of Ser. No. 09/698,307, filed Oct. 29, 2000, which is a continuation-in-part of 09/618,328, filed Jul. 18, 2000, which is a continuation-in-part of Ser. No. 09/431,551 filed Oct. 29, 1999. The present application is a further continuation-in-part of Ser. No. 60/165,960, filed Nov. 17, 1999 and is also a continuation in part of Ser. No. 09/428,149, filed Oct. 27, 1999, and PCT/US00/29641 filed Oct. 27, 2000.

### FIELD OF THE INVENTION

The invention relates to an improved anti-adiposity anti-hypercholesterolemic dietary composition or pharmaceutical composition and a method for treating undesired adiposity in a mammal comprising the combined administration of an effective amount of a lipase inhibitor and an effective amount of a lipophilic polysaccharide. The lipase inhibitor and polysaccharide compositions may be administered in the same dosage form or may be administered separately within close time proximity. The composition and method give surprisingly improved synergistic results over their individual administration.

### BACKGROUND OF THE INVENTION

The absorption of fat found in food involves the cleavage of the primary ester bonds of triglycerides by a pancreas lipase into free fatty acids as well as mono- and diglycerides. These free forms of fatty acids as well as the mono- and diglycerides can be absorbed by the body and utilized as an energy source. Since the fatty acids and mono- and diglycerides are the most concentrated dietary forms of energy a great deal of calories result from the absorbed fat. If the intake of such calories is greater than the caloric needs “caloric level threshold” of the individual, then adiposity and even obesity can result.

To avoid undesired accumulation of absorbed fat and often a concurrent rise in cholesterol levels individuals have been encouraged to reduce their daily intake of fat. However, since dietary fat often provides a lot of the flavor for certain foods it is difficult to get individuals to continually comply with a low-fat diet over a long time period. A few alternatives have arisen to permit individuals to have a somewhat greater intake of fat than the amount dictated by their caloric level threshold, while minimizing the body's absorption and use of such fat that would result in excess calories beyond the caloric level threshold.

A popular lipase inhibiting drug known as Orlistat ((2S,3S,5S)-5-[(S)-2-formamido-4-methyl-valeryloxy]-2-hexyl-3-hydroxy-hexadecanoic 1,3 acid lactone) as well as other lipase or amylase inhibitors (see U.S. Pat. No. 5,643,874, which is incorporated herein by reference) have been used to inhibit lipases in the body and thereby prevent the absorption of dietary fat. At a 120 mg dose of Orlistat, taken before consuming a fat-containing meal (or up to one hour after eating such a meal), up to ⅓ of the fat eaten at a give meal will not be absorbed by the average person and utilized as fat calories. The undigested fat passes directly through the digestive system as an oil and is eliminated from the bowel in its oily undigested form. However, the undesired side effects of flatulence (wind) with discharge, oily discharge and oily or fatty stools are quite common while taking Orlistat or similar lipase inhibitors. Some meals such as those having fried foods greatly increase these undesired side effects since few people evenly distribute their intake of fat between all of the meals and snacks eaten in a given day. Of course, the solution for avoiding such undesired side effects is to restrict the amount of fat consumed and evenly distribute its intake over three meals each day, which essentially negates the reason for taking the lipase inhibitor. In fact, patients taking Orlistat are instructed by the manufacturer of Orlistat that there is no need to even take the medication if a meal is low in fat or fat-free. Therefore, patient compliance is difficult to obtain when a high-fat meal is consumed because of the resulting undesired side effects.

Another alternative to reducing fat intake is consuming the super fiber Chitosan which is a deacylated polysaccharide having the ability to absorb 6 to 8 times its weight in fat and oils. A combination of the polysaccharide with vitamin C (ascorbic acid) enhances the ability of the polysaccharide to absorb fat and cholesterol. The polysaccharide acts as a fat sponge and after absorbing the fat passes through the digestive system and is eliminated in the feces. The fat bound to the polysaccharide is also eliminated along with its caloric value. The polysaccharide chitosan is derived from a polysaccharide (chitin) obtained from the exoskeleton of shellfish such as shrimp or crabs, which is powdered and then chemically treated to deacylate the polysaccharide and thereby improve its ability to absorb fat. While the polysaccharide fiber from shellfish is similar to crude cellulose plant fiber, it has the ability to significantly bind fat in the digestive system as compared to plant fiber. Even in the presence of water the polysaccharide fiber from shellfish preferentially absorbs oil and fats. Since the polysaccharide fiber from shellfish is not digestible it has no caloric value. In order to be effective, the recommended dosage for Chitosan is about 1 gram per meal, which can be enough fiber to cause constipation, hard stool or other undesired side effects. Since the Chitosan ordinarily comes in 250 mg capsules, this would require taking at least 4 capsules with each meal, which [is] can discourage patient compliance.

U.S. Pat. No. 5,063,210 describes a class of non-absorbable sulfated water-soluble polysaccharides including chitin and chitosan and chitosan is more soluble than chitin. The sulfated compounds are all inhibit the cholesterol esterase.

The production of lipase inhibitors via fermentation processes produces a biomass or fermentation cake by-product which may be utilized as an animal foodstuff. To improve the consistency of the animal feed and the consistency of the resulting feces of the animals, a large amount of water insoluble crude plant fiber is added to the biomass, such as microcrystalline cellulose (AVICEL), wheat bran and oat bran (see, for example, U.S. Pat. No. 5,540,917). The amount of fiber suggested has a weight of 2 to 3 times the amount of fat consumed (weight ratio 1/1.5 of the animal feed, which is a hugh amount of fiber as the predominate portion of the food. This combination of biomass and crude plant fiber has the side effect of improving the activity level or potency of the lipase inhibitor enzyme in the biomass by product. U.S. Pat. No. 5,540,917 does not describe and give any examples of using non-plant fiber in combination with the biomass or fermentation cake byproduct.

Low fat, high fiber advocates have recommended a diet that is fueled by 10 to 20 percent fat calories and includes from 35 to 45 grams of fiber. Irrespective of intentions, most people are unable to consistently sustain this type of low-fat diet. Thus, lower fat absorption that can result in an effective absorption of from only 10 to 20 percent fat is very desirable. Improved compositions which can lower [a] fat absorption by up to ⅓ with minimal side effects are particularly desired, Accordingly, there is a need in the art for improved antiadiposity compositions and methods which do not require a low-fat diet to avoid or minimize undesired side effects of fiber or lipase inhibitors.

### SUMMARY OF THE INVENTION

The present invention relates to the concurrent or substantially concurrent administration of an amount of lipase inhibitor effective to reduce the absorption of dietary fat by up to about ⅓ of the consumed amount and a lipophilic polysaccharide in an amount from about 5 to 10 percent of the weight of the consumed fat. Examples of non-absorbable lipase and amylase inhibitors are described in U.S. Pat. Nos. 5,643,874 and 5,503,831, and Orlistat is a preferred lipase inhibitor. Examples of lipophilic polysaccharides such as Chitosan are described in U.S. Pat. No. 5,063,210 along with sulfated water soluble lipase inhibitors. Preferably, the lipase inhibitor and the polysaccharide are administered together, and more preferably in a single capsule. Alternatively, in another preferred aspect, the lipase inhibitor is administered in an effective amount and then from 1 to 4 250 mg capsules of the lipophilic polysaccharide (such as Chitosan) are taken separately and concurrently with the lipase inhibitor, with the number of capsules of the polysaccharide that are taken depending upon whether the meal has a small, medium or high fat content. Preferably from two to four of the 250 mg capsules of the lipophilic polysaccharide are taken along with the lipase inhibitor after a very high fat meal has been consumed.

### DETAILED DESCRIPTION OF THE INVENTION

### Preferred Embodiments

The present invention relates to the concurrent or substantially concurrent administration of an amount of lipase inhibitor effective to reduce the absorption of dietary fat to about ⅓ of the consumed fat and a lipophilic polysaccharide in an amount from about 2 to 10 percent of the weight of the consumed fat (about 100 mg to 1.5 g). Preferably, the lipase inhibitor is administered at about 50 to 150 mg per fat containing meal, and more preferably is Orlistat at about 120 mg. Preferably the lipophilic polysaccharide is administered at about 4 to 8 percent of the consumed fat, and most preferably from 5 to 6 percent of the consumed fat. For example, if a meal contains 15 grams of fat the lipophilic polysaccharide is preferably administered in an amount from about 250 mg to 1.5 g, more preferably 500 mg to 1 g, and most preferably from 500 to 750 mg.

Examples of non-absorbable lipase and amylase inhibitors are described in U.S. Pat. Nos. 5,643,874 and 5,503,831, which is incorporated herein by reference. Other non-absorbable lipase inhibitors having a similar type of lipase-inhibition activity to Orlistat (2S,3S,5S)-5-[(S)-2-formamido-4-methyl-valeryloxy]-2-hexyl-3-hydroxy-hexadecanoic 1,3 acid lactone may be utilized according to the invention. Orlistat is a preferred lipase inhibitor.

Examples of lipophilic polysaccharides such as Chitosan are described in U.S. Pat. No. 5,063,210 along with sulfated water soluble lipase inhibitors. Other water-soluble or water-insoluble Chitosan is the preferred lipophilic polysaccharide.

Preferably, the lipase inhibitor and the polysaccharide are administered together, and most preferably in a single capsule. The most preferred lipase inhibitor is Orlistat, and preferred administration is at the dose of 120 mg before (or within one hour after) a high-fat meal in combination with 250-500 mg of a lipophilic polysaccharide, (preferably Chitosan which is derived from the chitin of shellfish by deacylation).

In a preferred aspect, the lipase inhibitor is administered in an effective amount and from 1 to 4 250 mg capsules of Chitosan are taken concurrently with the lipase inhibitor, depending upon whether the meal has a small, medium or high fat content. Preferably from two to four of the 250 mg capsules of Chitosan are taken along with the lipase inhibitor after a very high fat meal has been consumed.

Such combination of the lipase inhibitor and the lipophilic polysaccharide has a desirable combination or synergistic effect.

Since about ⅓ of the consumed fat is not absorbed due to the lipase inhibition, such fat would ordinarily cause undesired side effects from a high fat meal. The combination of a relatively small amount of non-absorbable lipophilic polysaccharide to the lipase inhibitor removes or reduces many undesired side effects caused by lipase inhibition by since it can soak up the unabsorbed fat resulting from lipase inhibition as the unabsorbed fat moves through the digestive system.

Likewise, one of the discouraging factors with respect to taking only a lipophilic polysaccharide to absorb and remove undigested fat is the large number of capsules necessary to remove a significant amount of the dietary fat before the body has had time to absorb the consumed fat. However, the combination of a lipase inhibitor with a lipophilic polysaccharide greatly reduces the amount of lipophilic polysaccharide that would have been necessary to have removed the same amount of consumed fat as the amount of unabsorbed fat resulting from lipase inhibition.

Therefore, a combination of a lipase inhibitor and a relatively small amount of a lipophilic polysaccharide can increase patient compliance with respect to taking a lipase inhibitor. Also, this combination can permit a patient the enjoyment of consuming a moderate amount of fat in their diet as nonabsorbable fat while avoiding or reducing undesirable side effects caused by a lipase inhibitor. Further, the combination of the relatively small amount of a lipophilic polysaccharide with the lipase inhibitor surprisingly does not interfere with the action of the lipase inhibitor and provides surprisingly good results with respect to avoiding or reducing unwanted side effects of the lipase inhibitor. The oily feces and oily discharge ordinarily resulting from the lipase inhibitor when a moderate amount of dietary fat is consumed may be generally replaced by merely soft to normal feces by utilizing the small amount of lipophilic polysaccharide. The use of such an amount of non-absorbable lipophilic polysaccharide also avoids the need for frequent bowel elimination or avoids the constipation that can occur with the ingestion of large of amounts of non-absorbable lipophilic polysaccharide fiber.

### Therapeutic Methods

In one aspect the present invention provides a method for treating adiposity or obesity comprising administering either concurrently or within about one hour an amount of lipase inhibitor effective to reduce the absorption of dietary fat by one tenth to about one third of the amount of fat consumed and a non-absorbable lipophilic polysaccharide in an amount from about 2 to 10 percent of the weight of the consumed fat, wherein each of the lipase inhibitor and the lipophilic polysaccharide are administered either prior to, or within one hour after, the consumption of a meal contain fat.

In a preferred aspect the lipase inhibitor administer is present in an amount effective to reduce such fat absorption by one fifth to about one third of the amount of fat consumed. Preferably, the lipase inhibitor is selected from the group consisting of orlistat, lipstatin, tetrahydrolipstatin and esterastin. More preferably, the lipase inhibitor is orlistat and is administered in an amount from 25 to 120 mg per fat-containing meal.

The lipophilic polysaccharide administered in such a method is preferably present in an amount of 4 to 8 percent of weight of the consumed fat. Preferably, the lipophilic polysaccharide administered in such a method is a member selected from the group consisting of chitosan, sulfated alginic acid, sulfated pectin, sulfated amylopectin, sulfated chitin, sulfated chitosan, sulfated dextran and sulfated nonsoluble plant cellulose. More preferably, the lipophilic polysaccharide is chitosan and is administered after a fat-contain meal in an amount from 4 to 8 percent of the weight of the consumed fat. Even more preferably, the chitosan is administered in an amount from 5 to 8 percent of the weight of the consumed fat.

In one embodiment the present invention provides a method wherein the lipase inhibitor and the lipophilic polysaccharide are administered in the same composition or dosage formulation.

In a second embodiment the present invention provides a method wherein the lipase inhibitor and the lipophilic polysaccharide are administered in separate dosage formulations.

### Compositions and Formulations

In one aspect the present invention provides a pharmaceutical composition for treating adiposity or obesity comprising an amount of lipase inhibitor effective to reduce the absorption of dietary fat by one tenth to about one third of the amount of fat consumed and a non-absorbable lipophilic polysaccharide in an amount from about 2 to 10 percent of the weight of the fat consumed in a meal.

Preferably, the lipase inhibitor is present in such a pharmaceutical composition in an amount effective to reduce such fat absorption by one fifth to about one third of the amount of fat consumed. The preferred lipase inhibitor is a member selected from the group consisting of lipstatin, tetrahydrolipstatin (orlistat), esterastin and tetrahydroesterastin. A lipase inhibit in a more preferred composition is orlistat, which is present in an amount from 25 to 120 mg per dose.

Likewise, preferably the pharmaceutical composition contains the lipophilic polysaccharide in an amount of 4 to 8 percent of weight of the fat consumed in a meal. Preferably, the lipophilic polysaccharide is a member selected from the group consisting of chitosan, sulfated alginic acid, sulfated pectin, sulfated amylopectin, sulfated chitin, sulfated chitosan, sulfated dextran and sulfated nonsoluble plant cellulose. More preferably, the lipophilic polysaccharide is chitosan and is present in an amount an amount from 4 to 8 percent of the weight of the consumed fat. Further preferred are such pharmaceutical compositions wherein the chitosan is present in an amount from 5 to 8 percent of the weight of the consumed fat.

More preferred are polysaccharide groups which are chitosan or other similar polysaccharide derivatives that have been modified by an ether group or one of the amino group and the modifying group is terminated by an organic acid group. Further preferred are modified chitosan polysaccharides wherein the organic acyl groups are independently a straight or branched chained alkanoyl group. Most preferred are such modified chitosan polysaccharide groups wherein the polarity resulting from the modification of chitosan with organic acyl groups allows the modified chitosan to absorb both lipids and water and to form a substantially homogenous gel with oil and water. Such polysaccharides are described in one or more of the parent applications from which this application claims priority, all of which are incorporated herein in their entirety by reference hereto.

In one embodiment, the present invention provides a composition wherein the lipase inhibitor and lipophilic polysaccharide are present in weight proportions of from 1:4 to 1:10 with respect to one another wherein the 1 corresponds to the weight of the lipase inhibitor. Preferably, the lipase inhibitor and lipophilic polysaccharide are present in weight proportions of from 1:5 to 1:8. Further preferred are such compositions in the weight proportions of about 1:6.

The compounds of this invention will typically utilize formulations such as a solution, a suspension, a hydrolyzable powder, a tablet or a capsule. The compositions of this invention may be administered in dosages that will provide optimal efficacy. The dose and method of administration will vary from subject to subject and be dependent upon such factors as the type of mammal being treated, its sex, weight, diet, concurrent medication, overall clinical condition, the particular compounds employed, the specific use for which these compounds are employed, and other factors which those skilled in the medical arts will recognize.

Formulations of the compounds of this invention are prepared for storage or administration by mixing the compound having a desired degree of purity with physiologically acceptable carriers, excipients, stabilizers etc., and may be provided in sustained release or timed release formulations. Acceptable carriers or diluents for therapeutic use are well known in the pharmaceutical field, and are described, for example, in Remington's Pharmaceutical Sciences, Mack Publishing Co., (A. R. Gennaro edit. 1985). Such materials are nontoxic to the recipients at the dosages and concentrations employed, and include buffers such as phosphate, citrate, acetate and other organic acid salts, antioxidants such as ascorbic acid, low molecular weight (less than about ten residues) peptides such as polyarginine, proteins, such as serum albumin, gelatin, or immunoglobulins, hydrophilic polymers such as polyvinylpyrrolidinone, amino acids such as glycine, glutamic acid, aspartic acid, or arginine, monosaccharides, disaccharides, and other carbohydrates including cellulose or its derivatives, glucose, mannose or dextrins, chelating agents such as EDTA, sugar alcohols such as mannitol or sorbitol, counterions such as sodium and/or nonionic surfactants such as Tween, Pluronics or polyethyleneglycol.

Dosage formulations of the compounds of this invention to be used for therapeutic administration must be sterile. Sterility is readily accomplished by filtration through sterile membranes such as 0.2 micron membranes, or by other conventional methods. Formulations can be stored in lyophilized form or as an aqueous solution. The pH of the preparations of this invention typically will be between 3 and 11, more preferably from 5 to 9 and most preferably from 7 to 8. It will be understood that use of certain of the foregoing excipients, carriers, or stabilizers will result in the formation of salts. The preferred route of administration is by oral administration as a capsule or tablet, but other methods of administration are also anticipated.

Therapeutically effective dosages may be determined by either in vitro or in vivo methods. For each particular lipase inhibitor compound or lipophilic polysaccharide of the present invention, individual determinations may be made to determine the optimal dosage required. The range of therapeutically effective dosages will naturally be influenced by the diet of the patient, the therapeutic objectives, and the ability of the patient to comply with a fixed or flexible dosage regimen. For example, once a desired weight has obtained, it may be desirable to provide a lower maintenance dose of the lipase inhibitor as a therapeutic dose which will permit the absorption of more than ⅔ of the consumed dietary fat, with a concomitant lowering of the amount of co-administered lipophilic polysaccharide, e.g., 25 to 75 mg of the lipase inhibitor before meals and from 100 to 750 mg of the lipophilic polysaccharide. Accordingly, it may be necessary for the therapist to vary the dosage and modify the frequency of administration of the composition for a patient to obtain an optimal therapeutic effect. The determination of effective dosage levels, that is, the dosage levels necessary to achieve the desired result, will be within the ambit of one skilled in the art. Typically, applications of compound are commenced at lower dosage levels, with dosage levels being increased until the desired effect is achieved.

Typically, the lipase inhibitor or a mixture of such inhibitors in an amount from about 0.5 to 500 mg as the free acid or base form or as a pharmaceutically acceptable salt, is compounded with a physiologically acceptable vehicle, carrier, excipient, binder, preservative, stabilizer, dye, flavor etc., as called for by accepted pharmaceutical practice. The amount of active ingredient in these compositions is such that a suitable dosage in the range indicated is obtained. Similarly, capsules or tablets containing from 100 mg to 500 mg of the lipophilic polysaccharide can be formed. In one embodiment, 25 mg to 120 mg of the lipase inhibitor is combined with from 100 to 500 mg of the lipophilic polysaccharide and optionally compounded with a physiologically acceptable vehicle, carrier, excipient, binder, preservative, stabilizer, dye, flavor etc., as called for by accepted pharmaceutical practice. Such resulting combination may be obtained in capsules or pill for containing from 125 to 620 mg of the combination active ingredients.

Typical adjuvants which may be incorporated into tablets, capsules and the like are a binder such as acacia, corn starch or gelatin, and excipient such as microcrystalline cellulose, a disintegrating agent like corn starch or alginic acid, a lubricant such as magnesium stearate, a sweetening agent such as sucrose or lactose, or a flavoring agent. Other materials of various types may be used as coatings or as modifiers of the physical form of the dosage unit. Buffers, preservatives, antioxidants and the like can be incorporated according to accepted pharmaceutical practice.

In practicing the methods of this invention, the compounds of this invention may be used alone or in combination, or in combination with other therapeutic or diagnostic agents. In certain preferred embodiments, the compounds of this invention[s] may be co-administered along with other compounds typically prescribed for these conditions according to generally accepted medical practice, such as vitamin C or cholesterol synthesis blockers (such as lovastatin and the like). The compounds of this invention can be utilized in vivo, ordinarily in mammals such as primates, such as humans, sheep, horses, cattle, pigs, dogs, cats, rats and mice, or in vitro.

The compounds of this present invention, selected and used as disclosed herein, are believed to be useful for preventing or treating a condition characterized by undesired adiposity. In addition to the treatment of obesity, the compositions or active substance combination, in accordance with the invention, can be used for the treatment and prevention of overweight, such as diabetes, hypertension, hyperlipidemia and insulin-resistance syndrome.

In the case of all of these indications, the active substances can be used in the dosage ranges given above, with the individual dosage depending on the nature of the illness to be treated as well as on the age and condition of the patient and can be determined within the purview of the medical specialist.

Without further description, it is believed that one of ordinary skill in the art can, using the preceding description, make and utilize the compositions of the present invention and practice the claimed methods. The examples of lipase inhibitors and lipophilic polysaccharides as well as their therapeutic proportions, specifically point out preferred embodiments of the present invention, and are not to be construed as limiting in any way the remainder of the disclosure. Such examples are non-limiting in that one of ordinary skill (in view of the above) will readily envision other permutations and variations on the invention without departing from the principal concepts. Such permutations and variations are also within the scope of the present invention.

What is claimed is:

1. A method of treating adiposity or obesity comprising administering either concurrently or within about one hour an amount of lipase inhibitor effective to reduce the absorption of dietary fat by one tenth to about one third of the amount of fat consumed and a non-absorbable lipophilic polysaccharide in an amount from about 2 to 10 percent of the weight of the consumed fat, wherein each of the lipase inhibitor and the lipophilic polysaccharide are administered either prior to, or within one hour after, the consumption of a meal contain fat.

2. The method of claim 1 wherein the lipase inhibitor is present in an amount effective to reduce such fat absorption by one fifth to about one third of the amount of fat consumed.

3. The method according to claim 1 wherein the lipophilic polysaccharide is present in an amount of 4 to 8 percent of weight of the consumed fat.

4. The method according to claim 1 wherein the lipase inhibitor is selected from the group consisting of orlistat, lipstatin, tetrahydrolipstatin and esterastin.

5. The method according to claim 4 wherein the lipase inhibitor is orlistat and is administered in an amount from 25 to 120 mg per fat-containing meal.

6. The method according to claim 1 wherein the lipophilic polysaccharide is a member selected from the group consisting of chitosan, sulfated alginic acid, sulfated pectin, sulfated amylopectin, sulfated chitin, sulfated chitosan, sulfated dextran and sulfated nonsoluble plant cellulose.

7. The method according to claim 6 wherein the lipophilic polysaccharide is chitosan or a modified Chitosan and is administered after a fat-contain meal in an amount from 4 to 8 percent of the weight of the consumed fat.

8. The method according to claim 7 wherein the modified Chitosan is administered in an amount from 5 to 8 percent of the weight of the consumed fat and the modified Chitosan is modified with a sufficient number of organic acyl groups to cause the modified Chitosan to absorb or associate both lipids and water and to form a substantially homogenous gel with oil and water.

9. A method according to claim 1 wherein the lipase inhibitor and the lipophilic polysaccharide are administered in the same composition or dosage formulation.

10. A method according to claim 1 wherein the lipase inhibitor and the lipophilic polysaccharide are administered in separate dosage formulations.

11. A pharmaceutical composition for treating adiposity or obesity comprising an amount of lipase inhibitor effective to reduce the absorption of dietary fat by one tenth to about one third of the amount of fat consumed and a non-absorbable lipophilic polysaccharide in an amount from about 2 to 10 percent of the weight of the consumed fat, and said polysaccharide is capable of absorbing fat and dispersing the fat in an aqueous solution.

12. The composition according to claim 11 wherein the lipase inhibitor is present in an amount effective to reduce such fat absorption by one fifth to about one third of the amount of fat consumed.

13. The composition according to claim 11 wherein the lipophilic polysaccharide is present in an amount of 4 to 8 percent of weight of the consumed fat.

14. The composition according to claim 11 wherein the lipase inhibitor is selected from the group consisting of lipstatin, tetrahydrolipstatin, esterastin and tetrahydroesterastin.

15. The composition according to claim 11 wherein the lipase inhibitor is orlistat and is present in an amount from 25 to 120 mg.

16. The composition according to claim 11 wherein the lipophilic polysaccharide is a member selected from the group consisting of chitosan, sulfated alginic acid, sulfated pectin, sulfated amylopectin, sulfated chitin, sulfated chitosan, sulfated dextran and sulfated nonsoluble plant cellulose.

17. The composition according to claim 16 wherein the lipophilic polysaccharide is a modified chitosan and is present in an amount an amount from 4 to 8 percent of the weight of the consumed fat, wherein the modified Chitosan is capable of binding fat and then dispersing the absorbed fat in an aqueous solution.

18. A composition according to claim 11 wherein the lipase inhibitor and lipophilic polysaccharide are present in weight proportions of from 1:4 to 1:10.

19. A composition according to claim 19 wherein the lipase inhibitor and lipophilic polysaccharide are present in weight proportions of from 1:5 to 1:8.

20. A composition according to claim 18 wherein having from 25 to 75 mg of the lipase inhibitor and from 100 to 750 mg of the lipophilic polysaccharide.

21. A method according to claim 1 comprising administering a lipophilic polysaccharide comprising from 250 to 1000 mg of a modified chitosan prior to consuming a fat containing meal, administering concurrently with said fat containing meal, or within one hour thereafter, a separate dosage formulation of a lipase inhibitor comprising from 25 to 120 mg of orlistat, and adminstering in a separate dosage formulation currently with said fat containing meal, or within one hour thereafter an amount of chitosan to provide a overall amount of chitosan which is from 2 to 10 percent by weight of the fat consumed per meal, wherein the modified Chitosan is capable of absorbing fat and dispersing the absorbed fat in an aqueous solution.

22. The method according to claim 21, wherein for said fat containing meal the amounts of orlistat and modified chitosan administered are in weight proportions of from 1:4 to 1:10.

23. The method according to claim 21, wherein for said fat containing meal the amounts of orlistat and modified chitosan administered are in weight proportions of from 1:5 to 1:8.

24. The method according to claim 21, wherein for said fat containing meal the amounts of orlistat and modified chitosan administered are in the weight proportions of 1:6.

25. A method according to claim 21, wherein for said fat containing meal the amount of orlistat adminstered is 120 mg and the amount of modified chitosan administered is 1000 mg.
